(19) United States
(12) Patent Application Publication
(10) Pub. No.: US 2010/0074966 A1
(43) Pub. Date: Mar. 25, 2010

(54) Gaseous Mixture Containing O2 and N2O For Preventing Or Reducing Hyperalgesia

(75) Inventors: Gabriela Apiou, Paris (FR); Vincent Lagente, Rennes (FR); Thomas Marx, Paris (FR)

Correspondence Address: AIR LIQUIDE;Intellectual Property, 2700 POST OAK BOULEVARD, SUITE 1800, HOUSTON, TX, 77056, US

(73) Assignee: L'Air Liquide Societe Anonyme Pour L'Etude Et L'Ex ploitation Des Procedes Georges Claude, Paris (FR)

(21) Appl. No.: 12/594,496

(22) PCT Filed: Mar. 28, 2008
(86) PCT No.: PCT/FR2008/050549

§ 371 Date: Oct. 2, 2009

(30) Foreign Application Priority Data
Apr. 6, 2007 (FR) 0754341

(51) Int. Cl. A61K 33/00

(52) U.S. Cl. 424/600

(57) ABSTRACT

The invention relates to a gaseous mixture containing oxygen and nitrous oxide in a nitrous oxide proportion of between 5 and 15 vol. %, that can be used for producing a drug for preventing or reducing hyperalgesia in human beings or animals, in particular a post-operation opioid-induced hyperalgesia.

The present invention relates to a gas mixture containing oxygen (O₂) and nitrous oxide (N₂O) for preventing or minimizing/treating post-operative hyper-algesia in man or in animals.

During general anesthesia, it is customary to administer volatile or intravenous hypnotics to patients, or opioid or intravenous analgesic substances.

For example, mention may be made of the following anesthetic products which are commonly used in anesthesia, such as sevoflurane, desflurane, fentanyl, remifentanil or other opioids, etc.

However, it has been observed in practice that this induces undesirable effects in patients, known as “post-operative hyperalgesia”, which results in an increase in the sensitivity to pain of the patients, after the operation and/or the diagnostic phase during which the patient was anesthetized.

This post-operative hyperalgesia generates a greater sensitivity to pain whatever the origin or localization thereof, that is to say whether or not it is localized in the zone treated during the operation or the diagnostic phase.

This effect can be observed over a long period, which may range, in particular, up to 1 year, and results in a reduction in the quality of life of the individuals since they will be more sensitive to pain during this entire time.

One objective of the invention is to make it possible to avoid or at least to impede or reduce the appearance of this harmful post-operative hyperalgesia phenomenon in patients by providing an effective medicament that is capable of impeding or reducing the appearance of this phenomenon.

One solution according to the invention is a gas mixture containing oxygen (O₂) and nitrous oxide (N₂O) for preventing or minimizing/treating post-operative hyperalgesia in man or in animals, in which the proportion of nitrous oxide is between 5 and 15% by volume.

Within the context of the invention, the term “man” should be understood in its most general sense, namely “human being”, which therefore encompasses men, women and children.

Depending on the case, the gas mixture of the invention may comprise one or more of the following features:
it contains, in addition, oxygen or oxygen and nitrogen;
it is composed solely of oxygen and of nitrous oxide;
it is composed solely of oxygen, nitrogen and nitrous oxide, the volume proportion of oxygen being between 30 and 50%;
it preferably contains from 6% to 14% by volume of N₂O; and
it is packaged under a pressure greater than 2 bar within a pressurized container, in particular under a pressure greater than 90 bar in a gas cylinder.

In other words, the gas mixture containing oxygen and nitrous oxide according to the invention can be used as an inhalable medicament for preventing hyperalgesia in man.

Put another way, the invention also relates to the use of a gas mixture according to the invention for manufacturing an inhalable medicament for preventing or reducing hyperalgesia in man, in particular when the hyperalgesia is of post-operative type, in particular induced by at least one opioid.

The gas mixture of the invention is administered to the patient by inhalation, for example by means of a mask fed with gas or else by means of a respiratory ventilator, and this administration is carried out during the anesthesia phase of said patient.

The invention therefore also relates to a method of anesthetizing a patient comprising the following steps:
a) administering an opioid analgesic or anesthetic substance to a patient so as to obtain anesthesia of said patient by at least said substance;
b) keeping the patient under anesthetic during a given period, in particular during at least one part of the diagnostic phase and/or operating phase of said patient; and
c) administering to said patient, before, during and/or after step b), a gas mixture of O₂ and N₂O according to the invention, in particular a gas mixture containing oxygen (O₂) and nitrous oxide (N₂O) in a proportion of nitrous oxide between 5 and 15% by volume.

When it is desired to administer the N₂O/O₂ mixture of the invention to a patient via a ventilator, it will be possible to connect to said ventilator either a source of gas containing a pre-packaged gaseous mixture of O₂ and N₂O according to the invention, or a source of N₂O and a source of oxygen, and the N₂O/O₂ mixture, in the desired proportions according to the invention, will be made directly within said ventilator by mixing of said gases.

In other words, in the second case, the desired gas mixture will be achieved by adjustment of the ventilator and it is this which will automatically make the mixture by withdrawing the quantities of pure gas coming from each of the gas sources, such as gas cylinders or a network of pipes, or both, which are connected to it.

### EXAMPLE

The effectiveness of the gas mixture according to the invention was evaluated by administering a gas mixture containing 12.5% by volume of nitrous oxide, 50% oxygen and the remainder nitrogen to a group of 20 healthy voluntary individuals (test B) having received a volatile anesthetic, for example remifentanil, during a biomedical study involving an electrical stimulation model for 2 hours, which reproduces post-operative hyperalgesia.

The N₂O/O₂/N₂ gas mixture is administered by inhalation to the individuals by means of a respiratory mask connected to a source of the gas mixture, for example a gas cylinder containing the gas mixture.

By way of comparison, a placebo (gas formed from 50% oxygen and 50% nitrogen) was administered to the same group of 20 healthy voluntary individuals (test A) following strictly the same procedure.

The individuals, during test A and test B, are subjected to electrical stimulations so as to evaluate their degree of sensitivity to pain.

The hyperalgesia of these individuals was evaluated throughout the entire period of the electrical stimulation (10 measurements over 2 hours).

After comparing the results obtained, during test A, the healthy volunteers have a greater sensitivity to pain than during test B, when they have received the N₂O/O₂ mixture of the invention.

Owing to the N₂O/O₂ gas mixture of the invention, it is possible to effectively combat the harmful effect of opioid-induced post-operative hyperalgesia by administering said inhalable gas mixture to the airways of the patient.

The N₂O/O₂ gas mixture of the invention N₂O/O₂ may be administered before, during and/or after the opioid anesthesia phase, preferably before, during and after the anesthesia phase.

1-10. (canceled)

11. A gas mixture for use as an inhalable medicament for preventing or reducing hyperalgesia in man or in animals, said gas mixture comprising nitrous oxide and oxygen, the proportion of nitrous oxide being from 5 to 15% by volume.

12. The gas mixture of claim 11, wherein the hyperalgesia is of post-operative type.

13. The gas mixture of claim 11, wherein the hyperalgesia is of post-operative type induced by at least one opioid.

14. The gas mixture of claim 11, wherein the nitrous oxide is present in an amount from 6 to 14% by volume.

15. The gas mixture of claim 11, wherein the gas mixture consists essentially of nitrous oxide and oxygen.

16. The gas mixture of claim 11, wherein in addition to nitrous oxide and oxygen, the gas mixture contains nitrogen.

17. The gas mixture of claim 11, wherein the gas mixture consists essentially of oxygen, nitrogen and nitrous oxide with the volume proportion of oxygen being from 30 to 50% by volume.

18. The gas mixture of claim 17, wherein the gas mixture contains from 6 to to 14% by volume nitrous oxide.

19. An inhalable medicament for preventing or reducing postoperative hyperalgesia in man or in animals, said inhalable medicament in the form of a gas mixture and consisting essentially of nitrous oxide and oxygen, the proportion of nitrous oxide being from 5 to 15% by volume.

20. The inhalable medicament of claim 19, wherein the hyperalgesia is of post-operative type induced by at least one opioid.

21. An inhalable medicament for preventing or reducing postoperative hyperalgesia in man or in animals, said inhalable medicament in the form of a gas mixture and consisting essentially of nitrous oxide, oxygen and nitrogen, the proportion of nitrous oxide being from 6 to 14% by volume, the proportion of oxygen being from 30 to 50% by volume and the remainder being nitrogen.

22. The inhalable medicament of claim 21, wherein the hyperalgesia is of post-operative type induced by at least one opioid.

23. An inhalable medicament for preventing or reducing hyperalgesia in man or in animals, said inhalable medicament in the form of a gas mixture comprising nitrous oxide and oxygen, the proportion of nitrous oxide being from 5 to 15% by volume and said inhalable medicament packaged under a pressure greater than 2 bar within a pressurized container.

24. The inhalable medicament of claim 23, wherein the inhalable medicament is packaged under a pressure greater than 90 bar in a gas cylinder.
